(19) United States
(12) Patent Application Publication
(10) Pub. No.: US 2001/0051776 A1
(43) Pub. Date: Dec. 13, 2001

(54) Tinnitus masker/suppressor

(76) Inventor: Martin L. Lenhardt, Hayes, VA (US)

Correspondence Address: George E. Quillin, FOLEY & LARDNER, Washington Harbour, 3000 K Street, NW., Suite 500, Washington, DC, 20007-5109, US

(21) Appl. No.: 09/822,841

(22) Filed: Apr. 2, 2001

Related U.S. Application Data
(63) Continuation-in-part of application No. 09/417,772, filed on Oct. 14, 1999.
(60) Provisional application No. 60/104,233, filed on Oct. 14, 1998.

(51) Int. Cl. A61B 17/22

(52) U.S. Cl. 601/2

(57) ABSTRACT

A system and method for tinnitus masking or suppression. At least one upper audio frequency is provided to a head of a patient, to thereby stimulate the auditory cortex. The upper audio frequency is preferably applied by way of air conduction. At least one ultrasound frequencies can also be applied by way of bone conduction. Once stimulated, the auditory cortex will mask or suppress tinnitus.

### RELATED APPLICATIONS

This application is a continuation in part of U.S. patent application Ser. No. 09/417,772, filed Oct. 14, 1999 which itself claims priority to U.S. provisional patent application Ser. No. 60/104,233, filed Oct. 14, 1998, both of which are incorporated in their entirety herein by reference.

### BACKGROUND OF THE INVENTION

1. FIELD OF THE INVENTION

The present invention relates to a system and method for masking or suppressing tinnitus. In particular, the present invention relates to a system and method for masking or suppressing tinnitus using high frequency signals, such as upper audio signals in one embodiment and ultrasound and higher range signals in other embodiments, that affect the cortical auditory and other neurons in the brain.

2. DESCRIPTION OF THE RELATED ART

Tinnitus is defined as any ringing in the ears for which there is no external source. Tinnitus is considered a phantom sound, which arises in the brain and not actually in the ears as it appears to subjectively. For example, a ringing, buzzing, whistling, or roaring sound may be perceived as tinnitus. Tinnitus can be continuous or intermittent, and in either case can be very irritating to one who has such an affliction.

Prior to the present invention, there has been no consistently effective way to counter, or mask, tinnitus. Most of the attempts to date have focused on masking the perceived sound. For example, U.S. Pat. No. 4,222,393, issued to Robert Hocks et al., describes a tinnitus masker that provides sounds in the range of from 1000 Hz to 5000 Hz, with a peak around 3000 or 4000 Hz. The patient is provided with sounds of varying pitch, one after another, so that the patient can identify the particular external sound having the same pitch as the tinnitus that the patient is experiencing. Once this is done, a power operated sound is applied to the ear of the patient, with that sound including a range of frequencies extending in a range above and below the perceived pitch.

U.S. Pat. No. 4,226,248, issued to Samir Manoli, describes a phonocephalographic device, which is used to passively, non-invasively monitor sounds from the surface and cavities of a patient's head and correlate these sounds with a person's elecytrocardiagraph (ECG). A pair of insertable ear microphones of ample sensitivity are inserted into the patient's ears, where they detect sounds from the surface and cavities of the head. These signals are processed, with the processing including the filtering of these signals through a frequency analyzer, which is made up of four Butterworth filters with a variable center frequency of between 150 Hz and 1000 Hz. In addition, the output signals may be passed to a oscillator for display on an oscilloscope, and or may be displayed on a chart recorder. As such, this apparatus may be used to diagnose certain medical problems of the patient, including tinnitus.

U.S. Pat. No. 4,759,070, issued to Barry Voroba et al., describes a patient controlled master hearing aid. The device includes a hearing test module and an operator's and patient's console. Based on this testing apparatus, the patient can select electronic components to be employed in his or her hearing aid, which can be configured to address tinnitus. Testing and selection of a tinnitus masker are performed using a pseudo-random generator, which is connected to circuits through an analog switch.

U.S. Pat. No. 4,984,579, issued to Paul Burgert et al., describes a portable apparatus for treating afflictions of the ear. The apparatus temporarily changes the pressure in the ear canal to alleviate Meniere's symptoms, such as hearing loss, vertigo, tinnitus, nausea, and aural fullness, in which the patient can facilitate immediate self-treatment.

U.S. Pat. No. 5,024,612, issued to van den Honert et al., describes an external ear canal pressure regulating device and tinnitus suppression device. This device uses an in-the-canal external ear pressure-regulating device to alter the pressure of the fluid within the external ear canal. The device includes an earplug with a bulbous portion, which contacts the wall of the external ear canal and creates a seal that seals the external ear canal interior from the ambient environment. The earplug is inserted into the ear canal, and the bulbous end is compressed. Fluid is passed outwardly into the ambient environment through a valve, creating negative pressure in the exterior ear canal, which pulls the eardrum out. This decreases the pressure in the inner ear space. Once the bulbous end is released, it re-expands. This process can be repeated until the desired pressure differential, or tinnitus relief, is achieved.

U.S. Pat. No. 5,167,236, issued to Franz Junker, describes a tinnitus masker having an electric circuit arranged in a housing and an earpiece which produces a sound spectrum that masks the tinnitus. The sound spectrum contains a line spectrum with a fundamental tone, with an adjustment range of the fundamental tone of from 0.125 kHz to 20 kHz.

U.S. Pat. No. 5,325,827, issued to Saren Westermann, describes a tinnitus masker which uses one or more signal generators, a controllable amplifier, one or two electroacoustic transducers for converting the electrical signals into acoustic signals, and a voltage source. The signal generators generate a continuously repeated, sinusoidal pure tone signal which slowly moves through the audio frequency range and whose cycle duration can be adjusted between 0.1 and 1000 seconds.

U.S. Pat. No. 5,403,262, issued to Timothy Gooch, describes a minimum energy tinnitus masker, which produces a masking signal with a selected center frequency, selected bandwidth, and selected volume. The bandwidth selector allows for four selections, ⅛, ½, 1 octave bandwidth, as well as broad bandwidth; and the center frequency selector is selectable in a range of between 500 and 16,000 Hz.

U.S. Pat. No. 5,628,330, issued to George Upham, describes an apparatus for treating people who are afflicted with tinnitus. This apparatus includes an inner metal shell that is fitted onto a patient's head. The inner metal shell is nestled with a larger outer shell of similar characteristics. The patient experiences relief from tinnitus by holding an open end of the apparatus against the afflicted ear. The inventor of the ‘330 patent believes that his apparatus may focus or somehow direct the “natural healing process” of the human body to the injured part of the inner ear and/or direct external healing to the injured part of the inner ear. See column 4, lines 1-6.

U.S. Pat. No. 5,697,975, issued to Matthew Howard III, et al., describes a human cerebral cortex neural prosthetic for tinnitus. Howard's device can be positioned in the brain so that electrical discharges can be accurately transmitted to geometrically dispersed locations in either a cortex or the thalamus, to allow a physician to physiologically test location and function of the neural prosthetic electrodes to reduce/eliminate the patient's tinnitus. In this regard, Howard's invention treats tinnitus in the brain, and not in the inner ear. In particular, Howard describes that the normal transduction of sound waves into electrical signals occurs in the cochlea, which is a part of the inner ear located within temporal bone. The cochlea is tonotopically organized, which means that different parts of the cochlea respond optimally to different tones. One end of the cochlea (base) responds best to high frequency tones, while the other end (apex) responds best to low frequency tones. The cochlea converts the tones to electrical signals, which are then received by the cochlea nucleus in the brain. This converted information is passed from the cochlea into the brain stem by way of electrical signals carried along the acoustic nerve, and in particular, the cranial nerve VIII. As the acoustic nerve leaves the temporal bone and enters the skull cavity, it penetrates the brain stem and relays coded signals to the cochlear nucleus, which is also tonotopically organized. Through many fiber-tract interconnections and relays, sound signals are analyzed at sites throughout the brain stem and the thalamus, with the final signal analysis site being the auditory cortex situated in the temporal lobe of the brain.

U.S. Pat. No. 5,663,727, issued to Peter Vokac, describes a frequency response analyzer and shaping apparatus, and digital hearing enhancement apparatus. The device provides many of the characteristics of a complete fast fourier transform suitable for audio signals and other signals. Vokac's device customizes the frequency response for a particular patient, by providing an FFT'ed signal in an audible frequency range.

U.S. Pat. No. 5,692,056, issued to William Gardner, describes a method and apparatus for intracranial noise suppression. Vibrations from an instrument, as well as vibrations in the bone structure of the patient, are sensed and processed to generate canceling noise, which is then fed into the inner ear through vibrations on the head. Gardner's device also includes an equalizer and an automatic adaptive coupler.

Also, there is on the market an electrical tinnitus suppressor called “Theraband™”. This is a battery powered headset that delivers amplitude modulated radio frequency waves to the subject. The carrier is about 60 kHz (possibly variable), with audio frequencies in the 200 Hz to 20,000 Hz range. The means of delivery is to the ear of the subject, where the sounds are received like any other sound. Theraband™ uses electrical energy capacitively coupled to the head via electrodes on mastoid.

All of the above-mentioned tinnitus maskers do not appear to fully mask tinnitus, since they do not appreciate the true reason why tinnitus occurs. In particular, these conventional tinnitus maskers/suppressors operate under the assumption that the tinnitus problem is in the inner ear, and they attempt to provide a solution that is based on this assumption.

### SUMMARY OF THE INVENTION

The invention is directed to a tinnitus masker/suppressor, which includes an upper audio source configured to output at least one upper audio frequency. The masker/suppressor also includes an output unit connected to the upper audio source and configured to convert the upper audio frequency to an output signal to be provided to the patient via air conduction. The output signal provides a stimulation of the brain of the patient, which in turn causes tinnitus masking or suppression.

The invention is also directed to a method of masking tinnitus, which includes a step of providing at least one upper audio frequency to a head of a patient.

The invention is further directed to a method of examining a patient in order to provide a treatment for that patient. The method includes a step of providing a plurality of upper audio frequency tones, in sequence, to the patient, to determine an optimum ultrasound frequency for the patient. The method also includes a step of providing a plurality of audible frequencies modulated by the determined optimum upper audio frequency, so as to determine a particular audible frequency that is optimum for the patient with respect to tinnitus masking or suppression.

### BRIEF DESCRIPTION OF THE DRAWINGS

The above-mentioned object and advantages of the invention will become more fully apparent from the following detailed description when read in conjunction with the accompanying drawings, with like reference numerals indicating corresponding parts throughout, and wherein:

FIG. 1 is a block diagram of a tinnitus masker according to first and second embodiments of the invention;

FIG. 2 is a block diagram of a tinnitus masker according to a third embodiment of the invention;

FIG. 3 is a diagram showing a brain-sphere model used to determine resonant frequencies of a brain;

FIG. 4 shows one possible transducer that may be used to provide bone stimulation to the patient, so as to treat tinnitus in accordance with embodiments of the invention;

FIGS. 5A and 5B show the lower two-most resonance frequencies obtained by using the transducer of FIG. 4;

FIG. 6A is a plot of signal strength due to air load for a swept tone from 5 kHz to 250 kHz;

FIG. 6B is a plot of signal strength due to mastoid load for a swept tone from 5 kHz to 250 kHz; and

FIG. 7 shows elements used in a fifth embodiment of the invention, in which music is used to mask or suppress tinnitus.

### DETAILED DESCRIPTION OF THE PREFERRED EMBODIMENTS

The embodiments of the invention are directed to a method and a system for masking tinnitus, and may even suppress tinnitus. The incidence of tinnitus increases with age, affecting almost half of the population over seventy. Tinnitus is believed to exist in around 15% of the population. See 1989 National Strategic Research Plan, published by the National Institutes of Health, and referred to in U.S. Pat. No. 5,697,975, discussed in the Background section. Tinnitus is very often associated with hearing loss and noise exposure. Tinnitus can be described as a phantom sound (e.g., whistling, buzzing) that arises without any external stimulation. Often the source of tinnitus is assigned to the ear because it “sounds” like a sound, that it has the pitch, loudness and timbre of a sound. Tinnitus can be matched in quality to an external sound, and it is often associated with one ear or the other, or both ears. Tinnitus can often be masked by an external sound, as discussed in the Background of the Invention section of this application. There have been reports that, with the withdrawal of masking, tinnitus does not immediately reappear. This is termed tinnitus suppression. Suppression is typically short lived, and masking may again be required. The suppression phenomena is valuable in that masking may only be required for part of the day, such as for a short period of time in the morning, with the rest of the day being “tinnitus free” due to tinnitus suppression.

The fact that tinnitus is maskable suggests to most researchers that the source of tinnitus is in the ear to which it is localized. If this were true, then tinnitus masking would be nearly 100% effective using the method and apparatuses discussed in the Background of the Invention section, which is not the case. In fact, the matching of tinnitus with an external sound can be very difficult and is often unreliable. This had lead some to refine the masking energy in both spectrum and intensity, so-called minimum level of masking.

Alternatively, there are some researchers that pose a central origin to tinnitus, with that central origin being beyond the ear and in the brain. For example, an article by Lockwood et al., published in 1998, found widespread activation of the primary cortex contralateral to the ear as being the source of tinnitus. In other words, the source of tinnitus is actually cortical and not in the ear. This is a reasonable view since it has been demonstrated that auditory cortical neuron reprogramming in the ear is not capable of providing frequency-specific stimulation. The reprogramming process may well produce tinnitus as a by-product. Perceptually, the source of cortical stimulation is directed to the peripheral sensory end organ. The reason for failure of attempts to mask or pharmaceutically treat tinnitus in the ear may well be that the ear is not the site of tinnitus!

This view of having a central origin for the source of tinnitus is supported by the lack of success with conventional tinnitus maskers, and also with the observations that after surgically severing the auditory nerve, tinnitus persists, and further with position emission tomography (PET) scans. The neural imaging data show that tinnitus activates the primary auditory cortex contralateral to the ear in which the tinnitus is localized, with that area activated being broader than that activated by sounds of similar frequency. This is one important reason why conventional tinnitus maskers fail, since they do not completely mask the tinnitus at the central origin or location. To broaden the frequency spread at the cortex, a masking signal that is broader and louder at the ear must be provided. However, when such a signal is given to patients who suffer from tinnitus, they find that the masker is more intolerable than the tinnitus. In other words, the cure is worse than the disease.

To determine a better cure for tinnitus, one has to understand the workings of the inner ear and the brain. External sounds activate both primary cortices, and each cortex is connected to a respective ear via a descending auditory nervous system. Maskers have an additional limitation in that if fitted on the left ear due to tinnitus localized left, both auditory cortices are stimulated, even though only the right cortex is activated by the tinnitus. The masker will in fact interfere with normal auditory function in the brain, and this will contribute to patient intolerance and discomfort. The brain will actively try to reduce the amount of masking arising up the auditory pathway by activating the descending auditory neural track. The result is that the brain will try to turn down the masker, limiting its effectiveness.

As a result, what is needed is a stimulus that is sufficiently salient to mask the tinnitus, but is not treated as an unwanted signal that will be inhibited by the brain. A masker that provides such a stimulus will be effective in terms of auditory cortical activation, and will not interfere with everyday important sounds, such as speech. Such a masker will be effective with people having hearing loss.

While there may be disagreement about the site of tinnitus (ear versus brain), most researchers agree that tinnitus and hearing loss are linked. Although documentation is incomplete, some deaf individuals also complain of bothersome tinnitus. Conventional tinnitus maskers are not very effective with those persons who have profound hearing loss. Also, it is desirable to have a masker that is audible only to the patient and does not radiate into the environment. Maskers that are implanted into the middle ear fit this criterion, but other types of maskers do not.

The masking stimulus that will meet all of the above criteria, and that is used in the tinnitus masker and method according to several embodiments of the invention, is ultrasonic noise. This noise can be made up of any part of the spectrum from 20,000 Hz up to 200,000 Hz. In a second embodiment, the noise band may extend from 10,000 Hz to 200,000 Hz. In a third embodiment, frequencies in an imaging frequency band of from 200,000 Hz to 5 MHz may be used with or without the other ranges in the first two embodiments. Alternatively, single tones in the ranges provided in the first through third embodiments may be used instead of noise. In a fourth embodiment, a single tone or noise in a range of from 10 kHz to 20 kHz may be used, whereby this frequency range corresponds to an upper audio frequency range.

There have been two reports of ultrasonic tinnitus suppression in the literature: Carrick et al., 1986 British Journal of Audiology, vol. 20, pages 153-155; and Rendell et al., 1987 British Journal of Audiology, vol. 21, pages 289-293. The Carrick article reported positive findings using a 500 kHz pulsed ultrasonic suppressor that produced 57 kPa of energy at 1 cm with 4 mW cm² of power. The Rendell article failed to replicate those findings using the same equipment and drawing subjects from the same clinic population. This technique appears to have been abandoned.

Pulsed ultrasound in the low to mid kHz has been shown to introduce lower frequency transients into the signal. It is now believed that the low frequency ultrasound that was effective in tinnitus suppression in the above-mentioned studies. Since this feature was not presented optimally or perhaps consistently, varied positive results could be expected, as is the case with the differences in results in the two studies.

In the case the MHz tonal or noise frequencies used according to the third embodiment of the invention, the stimulus is preferably provided in a pulsed manner. The rate of pulsing is not critical, but a slow rate of pulsing, such as a rate from 1-10 Hz, is preferred. Because the tinnitus masker according to the embodiments of the invention is high pitched and broad in spectrum, the tinnitus-affected area of the cerebral cortex will virtually all be masked. Since the delivery intensity will be low, minimal energy (re: threshold) will be expended. Since ultrasound is difficult to detect by air conduction, the masker will be personal and inaudible to others who may be nearby the person undergoing tinnitus masking treatment. Since those with severe hearing loss can detect ultrasound, such as by using a supersonic bone conduction hearing aid as described in U.S. Pat. No. 4,982,434, which is incorporated in its entirety herein by reference, it will address their needs for a masker. Preliminary results suggest temporary tinnitus suppression by using an apparatus or method according to the embodiments of the invention.

The spectral energy that is provided to suppress tinnitus of from 10 kHz upward can be a single tone or filtered noise. It can be continuous or pulsed. The spectral energy is preferably delivered near or at no more than 20 dB or so above threshold (e.g., between threshold and 20 dB above threshold). Delivery is preferably by a vibrator placed on the skin of the head or neck. A MHz pulser, to be used to deliver MHz noise signals according to the third embodiment, will preferably be delivered to the skin over the foreman magnum (back of skull by the neck). A transducer will preferably be similar to that used in transcranial Doppler insonation.

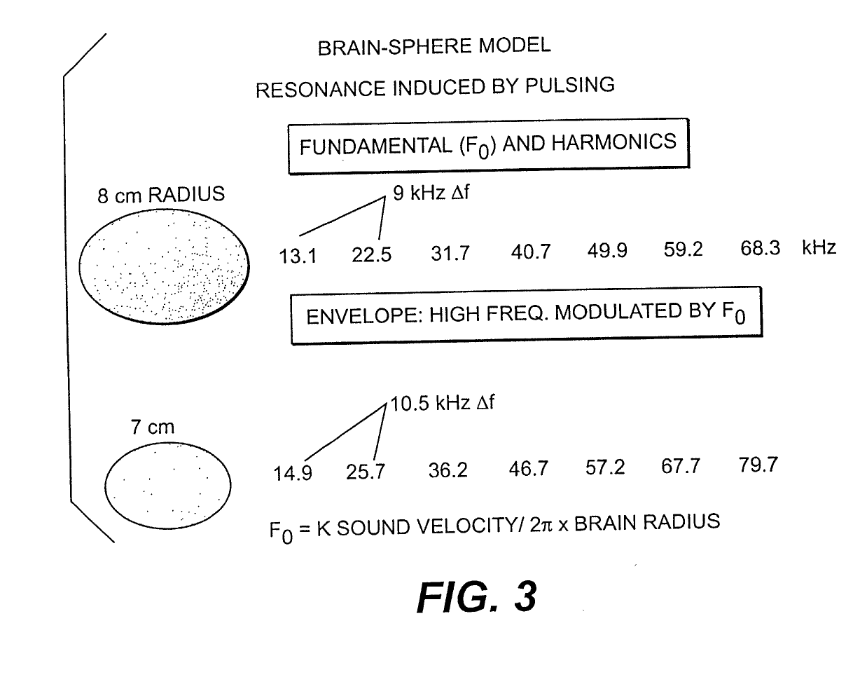

Ultrasound affects not only a wide area in the ear (sending afferent information to the auditory cortex), but it also affects the brain itself. Ultrasound actually pulses the brain since the brain's fundamental resonant frequency is in the low ultrasonic range to the high audio range (determined by the diameter of the brain and sound velocity in water). FIG. 3 shows a brainsphere model used to compute the brain's fundamental resonant frequency for two differently-sized brains. The computation of the brain's fundamental resonant frequency is based on the model of the brain as a sphere with the skull as a boundary. As a result, a number of resonant frequencies will be generated when the brain is pulsed.

Pulsed ultrasound of noise according to the third embodiment will also send the brain into oscillation at its resonant frequency, and thus is also a viable means of stimulation. Delgado and Monteagudo (1995) demonstrated that low frequency amplitude-modulated (am) ultrasound can effectively stimulate cortical neutrons, which was used to stimulate brain tissues for brain modification. The present invention also stimulates cortical neurons, but for the purpose of tinnitus masking, which was not proposed by Delgado and Monteagudo.

Therefore, several of the embodiments of the present invention provide for the use of ultrasound to mask tinnitus by stimulating any remaining high frequency area in the ear and by suppressing tinnitus by acting on cortical auditory neurons in the brain.

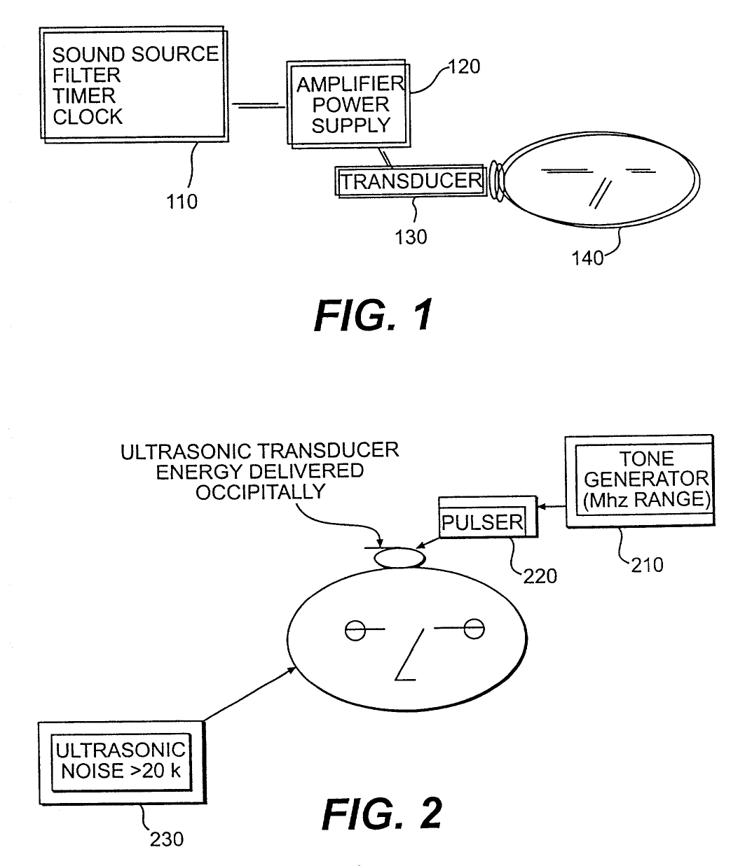

FIG. 1 shows a block diagram of an apparatus for tinnitus masking according to either the first or second embodiments of the invention. In FIG. 1, a sound source unit 110 produces filtered noise (over a range of frequencies) or a frequency tone. In the first embodiment, the ultrasonic energy is presented as an amplitude modulated carrier that can be set at any discrete frequency from 20 kHz to 200 kHz. The range can be set to any discrete frequency from 10 kHz to 200 kHz in the second embodiment, anywhere from 200 kHz to 5 MHz in the third embodiment, and anywhere from 10 kHz to 20 kHz in a fourth embodiment. The carrier also may be swept over the entire range or part thereof. The carrier is multiplied by an audio tone in the range of from 1 kHz to 20 kHz. This corresponds to a carrier modulated by audio. The audio tone can also be presented over a small range or swept through the entire range of audio frequencies. Sweep time is variable, and preferably is set to a range of from 2 to 3 minutes. The flexibility in the carriers and audio frequencies allows an operator to set frequency parameters such that the end product is stimulation over the ultrasonic range of from 20 kHz to at least 200 kHz. Speech or music also may be employed as part of the audio frequencies.

The fourth embodiment uses an amplitude modulated carrier that is solely in the upper audio range in order to provide tinnitus masking or suppression. This embodiment has an advantage in that, due to the use of a lower frequency range, the power consumption is less than it is for the other frequency ranges used in the first, second and third embodiments. Also, in the fourth embodiment, the tinnitus treatment signal is provided to the patient via airborne conduction. Bone conduction may alternatively be used along with the air conduction method of providing the treatment signal, to get two different conduction paths in the fourth embodiment. For example, if a transducer is used to provide bone conduction, and at the same time sound is provided to the patient's ear by way of a CD (containing tinnitus treatment signals in accordance with the fourth embodiment) and headphones, the tinnitus is treated by way of these two different ways of providing the tinnitus treatment signals simultaneously to the source of the tinnitus within the patient's brain. Alternatively, only air conduction or only bone conduction may be used to provide the tinnitus treatment signals to the patient in the fourth embodiment.

The preferred method of signal transmission is by way of double sideband modulation (suppressed carrier). Full amplitude modulation (full am carrier plus both sidebands) or single sideband modulation (either upper or lower sideband with the carrier and the other sideband suppressed) can alternatively be utilized. Modulation depth preferably does not exceed 90%, and the energy does not exceed 15 kPa (in water at 3.5 cm). Total power is preferably limited to 30 mW cm². Commercially available piezoelectric transducers are used to deliver the ultrasound in vibratory form to the patient's head. The precise level of energy (not to exceed 15 kPa) is to be determined for each patient during testing of each patient. The ultrasound may be audible during therapy. In the fourth embodiment that utilizes air conduction, sound pressures will be maintained at or below comfortable listening levels and in compliance with federal safety standards on sound exposure.

Referring back to FIG. 1, the sound source unit 110 includes a filter for producing filtered noise, a timer, or clock. These elements operate as a pulse filter for ultrasonic noise, with the timer or clock providing the pulse timing. The output of the sound source unit 110 is provided to an amplifier and power supply unit 120, which amplifies the signal to the proper level to provide a signal to the patient at the low, minimal energy, as explained above. A transducer unit 130 converts the output of the power supply unit 120 to a vibration, which is felt by the patient. The transducer unit 130, preferably a piezoelectric device, is placed somewhere on the patient's head 140, preferably just behind the ear. Those vibrations are provided to the brain (not shown) within the skull of the patient's head 140, thereby stimulating the cortices and masking tinnitus.

FIG. 2 shows the differences between the delivery of ultrasound noise according to the first and second embodiments as compared to the third embodiment. In the third embodiment, a tone generator 210 provides a tone in the MHz range. The output of the tone generator 210 is provided to a pulser 220, which provides pulses of MHz noise at a predetermined rate, say, between 1 and 10 Hz rate. A transducer (part of the ultrasonic noise unit 230) is preferably situated on the patient's skin on the back of the skull by the neck. FIG. 2 also shows the delivery of non-pulsed ultrasonic noise in the range of from 20 kHz to 200 kHz via an ultrasonic noise unit 230. In FIG. 2, ultrasonic noise unit 230 includes the sound source unit, amplifier and power supply unit, and transducer unit shown in FIG. 1.

Thus, according to the embodiments of the invention, an ultrasonic transducer delivers energy occipitally to the patient, to thereby mask and/or suppress tinnitus.

The ultrasound technique discussed herein is not without some disadvantages. The ultrasound technique does not produce low frequency stimulation of the inner ear, as with the conventional electrical maskers. Some tinnitus is low pitched, and thus may not be masked by the ultrasound technique described herein, but most tinnitus is not in this range. The electrical signal provided by the conventional tinnitus maskers is presumably demodulated at the skin or cochlea, leaving the audio frequencies “in” the inner ear. However, the ultrasound technique according to the embodiments of the invention does not appear to demodulate in the cochlea. Rather, the energy focuses at the base of the cochlea, in the region that codes audio frequencies from 5,000 Hz upwards.

However, the embodiments have several advantages over conventional maskers, some of which have already been described. Low frequency neural synchronization can be accomplished with ultrasound when it is amplitude modulated by very low audio frequencies, for example, 1 Hz to 50 Hz. The precept is of high pitch sound having a low frequency periodicity. The periodicity can be increased or decreased by changes in the audio frequency tone. Thus, the ultrasound tinnitus suppression apparatus and method according to the embodiments of the invention provides only high frequency stimulation presumably in the area of damage (as indicated by the tinnitus pitch). Furthermore, auditory nerve low frequency synchronous firing can also be incorporated in the ultrasound treatment regime according to the embodiments of the invention.

According to the invention, the site of action in the inner ear appears to be the hair cells for MHz amplitude modulation, in which the audio tone is reintroduced by demodulation. In the ultrasound method and apparatus according to the invention, demodulation does not appear to take place in the cochlea, but instead the site of action appears to take place at the cilia of the hair cells. The cilia have ultrasonic resonance, and a movement of endolymph by a compressive intracochlear ultrasonic wave may have rejuvenative effects on the cell directly. Stimulation of nearby cells (with respect to those injured) will also stimulate adjunct areas in the central nervous system, which could activate inhibitory influences in the ear.

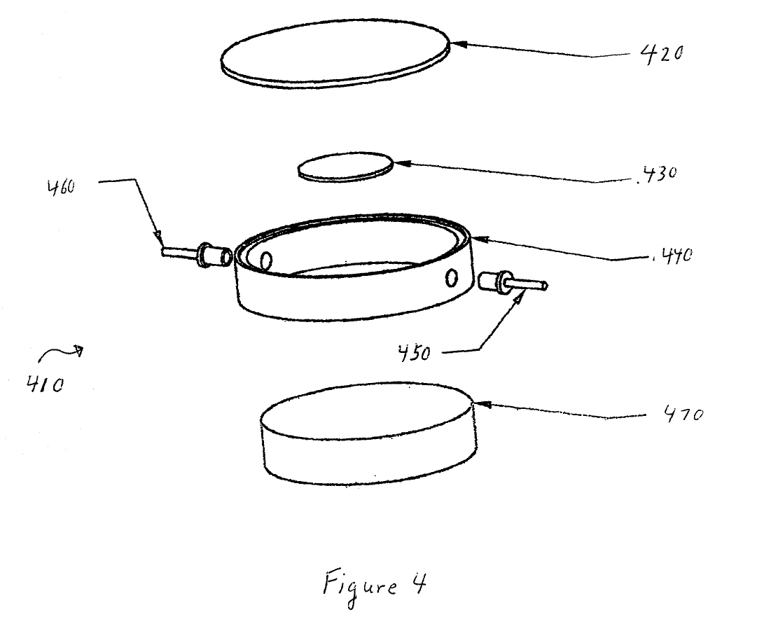

FIG. 4 shows the separate components making up a transducer 410 that can be utilized in any of the embodiments of the present invention, in order to provide a vibration to a patient's head or neck by way of bone conduction. The components are shown separately disposed from each other in order to provide a clear description of the transducer 410, whereby these components are coupled to each other to provide an integral transducer during a manufacturing process for making the transducer 410.

The transducer 410 includes an aluminum disk 420, a piezo (PZT) disk 430, an aluminum collar 440 (with a recess machined so as to receive the aluminum disk 420), a case ground solder pin 450, an insulated solder pin 460, and a foam rubber damping plug 470. Alternatively, the foam rubber damping plug 470 may be substituted with a vinyl cap. In a preferred construction of the transducer 410, the piezo disk is bonded to the aluminum disk with silver bearing epoxy, the aluminum disk is bonded into the recess of the aluminum collar with silver bearing epoxy, a single solder wire (not shown) is soldered between the edge of the piezo disk and the insulator solder pin, and the case ground solder pin is coupled to the aluminum collar using a swaging tool to ensure good electrical contact to the aluminum collar. The transducer 410 as shown in FIG. 4 corresponds to a Blatek 40 KHz air ultrasonic transducer. Other types of transducers may be utilized in the present invention in order to provide a vibration to the patient's head or neck by way of bone conduction.

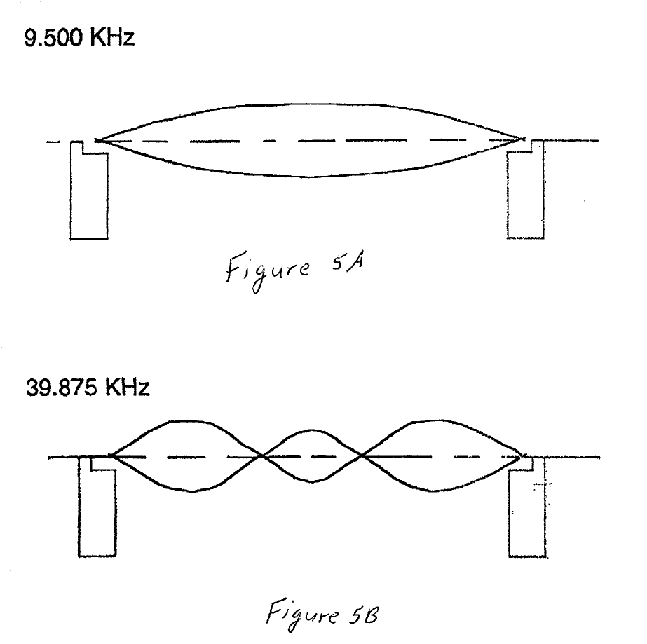

FIGS. 5A and 5b respectively show the first two resonances in air of the transducer 410 of FIG. 4. A first resonance is at 9.5 kHz, and a second resonance is at 39.875 kHz (approximately 40 kHz). The first resonance corresponds to a high audio frequency, and the second resonance corresponds to an ultrasonic frequency. Other resonances of the transducer of FIG. 4 occur at 97 kHz, 158 kHz, 206 kHz, and 240 kHz. These resonances can be varied by varying the transducer geometry, so as to obtain other resonances in the frequency range of interest in accordance with any of the embodiments of the present invention.

In the preferred configuration of the transducer utilized with the present invention, an oscillator (not shown) delivers a high ultrasound frequency, e.g., 200 kHz frequency, at low level to the transducer 410. The high ultrasound frequency activates, or stimulates, the vibratory motion such that less energy is required at frequencies near the fundamental and first harmonic to produce a useful amount of displacement at the skin (e.g., 1 micrometer displacement), than what would be required if the high ultrasound frequency was not provided to the transducer 410. An energy savings of about 15 volts has been achieved using a 200 kHz tone in conjunction with the audio or low ultrasonic frequencies that are supplied in accordance with the present invention so as to mask or suppress tinnitus. Of course, other high ultrasound frequencies besides 200 kHz may be utilized to achieve this energy savings (for example, using a high ultrasound frequency in the range of from 100 kHz to 500 kHz).

For patients that require less power to treat their tinnitus, a fifth embodiment of the invention inputs music, which is a form of pulsed stimulation. The music signal is filtered, and then multiplied by an upper audio signal, which corresponds to a carrier having a frequency value within the range of from 10 kHz to 20 kHz. The carrier can be tonal (single frequency between 10 kHz and 20 kHz) or noise (e.g., white noise between 10 kHz to 20 kHz, or a swept carrier in that frequency range). The music is pulsed in such a fashion as to be culturally agreeable to the listener, since music is (typically) meant to be enjoyed when heard. The output signal, which is the filtered music multiplied with the one carrier (or plurality of carriers, if more than one tone is used) in the range of from 10 kHz to 20 kHz, is not recognizable as music, but the output signal has the temporal or timbre of music.

In a preferred implementation of the fifth embodiment described above, the tinnitus stimuli are recorded on a compact disk (CD) with tracks varying in intensity level. The listener adjusts the volume of the stimulation by selecting the appropriate track of the CD. A relatively inexpensive CD player and headphones, plus the CD containing the tinnitus stimuli, are all that are required to treat the patient's tinnitus (which can be done anywhere—at work, at home, etc.). For example, the tracks may provide the stimulation in increasing volume of 1 dB increments. For example, tracks ranging from −54 dB to 0 dB may be provided, in six dB steps, on a single CD. Preferably, each track is of a duration of 1 minute and 25 seconds which can be looped for longer play time. Of course, other track durations are possible, while keeping within the spirit and scope of the invention. For example, track durations from as low as 10s of seconds to as much as 1 hour or more, may be contemplated. A standard CD player may be used to provide such treatment. All the user needs to do is to put the CD with the tinnitus masking/suppression signals according to the present invention into a CD player, and then put on his or her headphones. When the user turns the CD on to a particular track, the tinnitus masking or suppression treatment begins.

Tests performed using the present invention provide tinnitus masking or suppression for periods of two weeks or more, so that the patient can be treated with the tinnitus masker, and then not have to be retreated until weeks later. The masking effects linger for a period of time long after the tinnitus maskingtreatment according to the present invention has been performed on the patient.

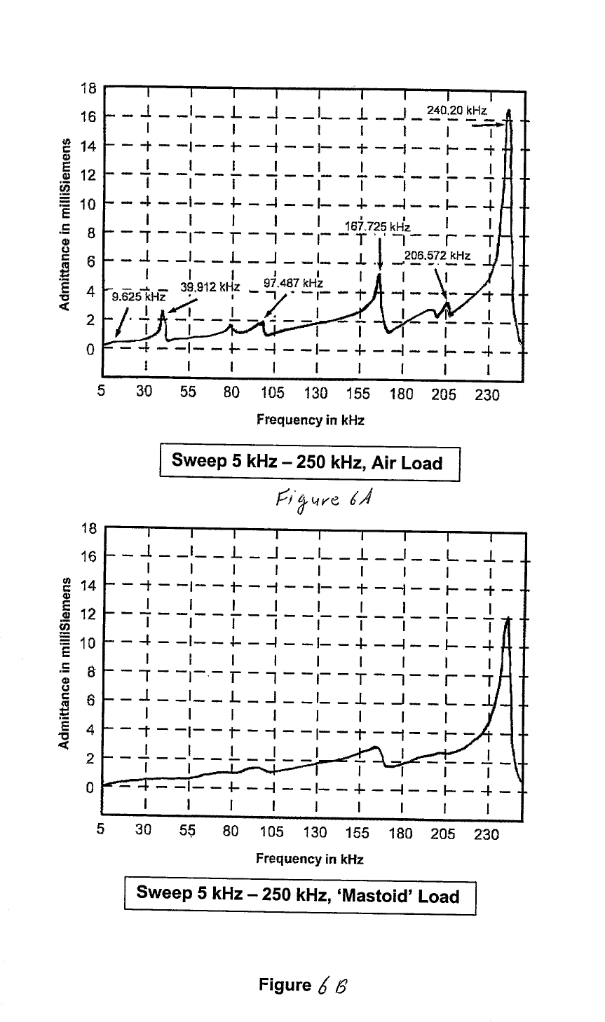

FIG. 6A is a plot of air load in the ear due to sweeping a treatment signal from 5 kHz to 250 kHz in accordance with embodiments of the invention, and FIG. 6B is a plot of mastoid load for the same range of frequencies. For the air load, peaks at 9.625 kHz, 39.912 kHz, 97.487 kHz, 167.925 kHz, 206.512 kHz, and 240.20 kHz were observed. For the mastoid load (which is the load on the temporal bone behind the ear), a peak at 240 kHz was observed. The resonances in air differ from those in the same transducer mass loaded by placing the transducer on the head.

For one example of utilizing music (or any complex acoustic pattern) with a carrier signal in order to provide a tinnitus masking signal, two tones are used as the carrier signal, one at 12 kHz and the other at 16.384 kHz. Of course, other frequencies or number of tones may be chosen within an acceptable range (e.g., 10 kHz to 20 kHz). The two frequencies are preferably chosen so as to better support music as an input signal. Music with an even spectral spread at a constant volume is the preferable type of music to use.

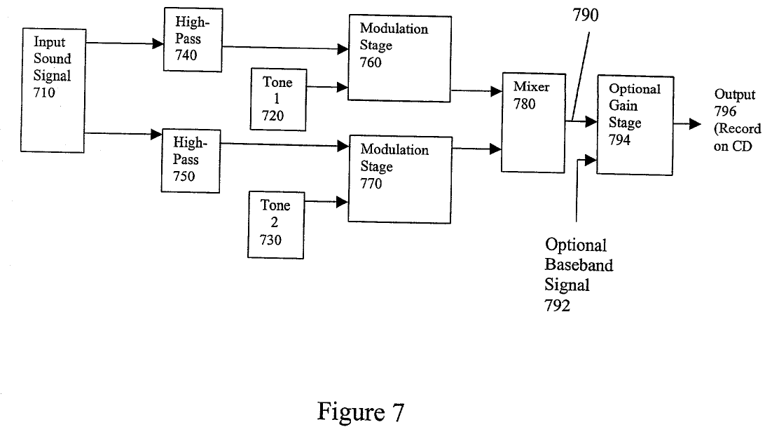

FIG. 7 shows one implementation for achieving a stimulus signal according to the fifth embodiment of the invention. The input signal 700, preferably music, is multiplied independently with a first tone 720 and a second tone 730 after having first been highpass filtered (e.g., using 1 kHz highpass filters 740, 750). The two filtered signals go through respective modulation stages 760, 770, one set 20 dB lower than the other (this value is adjustable, and can be set to a different value, such as between 10 to 30 dB in gain difference). The two gain-adjusted signals, after having passed through their respective modulation stages 760, 770, are then mixed together by mixer 780, and then highpass filtered by highpass filter 785 (e.g., an 8 kHz highpass filter), to obtain a signal 790.

As an optional element, a final adjustable gain stage 794 may be utilized to mix in some unprocessed baseband signal 792 with the signal 790, if desired. For example, a 200 kHz tone can be mixed with the signal 790 at optional gain stage 794. The 200 kHz tone activates the transducer that receives the output signal, to cause the transducer to operate at one of its higher resonance frequency modes. This results in less energy in the lower frequency range (e.g., processed noise) to be detected by the patient. The use of such a high frequency tone would not be utilized in the embodiments that use air conduction to provide the tinnitus masking/suppression signal to the patient.

The final output signal 796 is then recorded onto a CD, for playback through the tinnnitus treatment device or airborne through headphones. Thus, by using a high audio signal mixed with music, airborne conduction is achieved so as to provide some level of tinnitus masking or suppression. Also, bone conduction is also achieved, if a transducer, such as the one shown in FIG. 4, is also used to treat the tinnitus by being affixed to the patient's head or neck.

While preferred embodiments have been described herein, modification of the described embodiments may become apparent to those of ordinary skill in the art, following the teachings of the invention, without departing from the scope of the invention as set forth in the appended claims. For example, the pulsing as used in the third embodiment, may also be utilized in any of the other embodiments, so as to stimulate the brain at one or more of its resonant frequencies. Also, all of the components necessary to provide the tinnitus masking or suppression signals, may be accommodated on a single printed circuit board, to thereby make a fairly small-sized tinnitus masking or suppression device. For example, a printed circuit board may be used in accordance with the fourth embodiment. A signal output from the printed circuit board would be stored onto a CD, for playback on a CD player to treat a patient that has tinnitus.

Also, the fourth embodiment, which uses upper audio signals to treat tinnitus, may utilize a CD (and CD player and accompanying headphones) in order to provide the tinnitus treatment signals via airborne conduction to the auditory cortical neurons. The CD may be used with or without a separate transducer disposed on the neck or head of the user and that provides the tinnitus treatment signals by way of bone conduction. Instead of using a CD and a CD player, the tinnitus masking/suppression signals may be received by way of a network, such as the Internet, whereby patients access a particular web site, and download the tinnitus masking/suppression signals, such as in the form of an MP3 file, from a server. Once downloaded (after paying a fee to do so), the patient may play the MP3 file (to be provided to the patient via headphones connected to a personal computer that has downloaded the MP3 file, for example) to obtain treatment.

What is claimed is:

1. A tinnitus masker/suppressor, comprising:
an upper audio frequency source configured to output at least one upper audio frequency; and
an output unit connected to the upper audio frequency source and configured to convert the upper audio frequency to an output signal to be provided to the patient via air conduction,
wherein the output signal is used to mask or suppress the tinnitus.

2. The tinnitus masker/suppressor according to claim 1, further comprising an amplifier and power supply unit connected between the ultrasound unit and the output unit and configured to control an amplitude level of the at least one upper audio frequency to be no more than 20 dB greater than a threshold level of sound for the person.

3. The tinnitus masker/suppressor according to claim 1, wherein the at least one upper audio frequency is a frequency of between 10 kHz and 19.9 kHz.

4. The tinnitus masker/suppressor according to claim 2, wherein the at least one upper audio frequency is swept over a range of frequencies centered at the at least one upper audio frequency.

5. The tinnitus masker/suppressor according to claim 3, wherein the at least one upper audio frequency is swept over a range of frequencies centered at the at least one upper audio frequency.

6. A tinnitus masker/suppressor, comprising:
an input port for receiving an input sound in an upper audio range;
an ultrasound frequency source that outputs an ultrasound frequency;
a first gain stage that is configured to multiply the input sound with the ultrasound frequency, and to output an output signal that is further multiplied by a first gain value;
a recording medium that receives the output signal and that records the output signal for playback at a later time.

7. The tinnitus masker/suppressor according to claim 6, wherein the input sound is a music signal.

8. A method for treating tinnitus, comprising:
a) mixing an input sound signal with an upper audio frequency signal, to obtain a mixed signal;
b) recording the mixed signal onto a recording medium; and
c) treating a patient by providing the mixed signal to the patient using the recording medium, by way of air conduction.

9. The method according to claim 8, further comprising:
d) mixing an ultrasound frequency signal with the mixed signal, to obtain a second mixed signal,
wherein the second mixed signal is recorded onto the recording medium and provided to the patient to treat the patient.

10. A method of masking or suppressing tinnitus, comprising:
a) providing at least one upper audio frequency to a head of a patient by way of air conduction.

11. The method according to claim 10, wherein the noise is within a range of from 10 kHz to 19.9 kHz.

12. The method according to claim 10, further comprising:
b) pulsing the noise before applying the at least one upper audio frequency before applying it to the head of the patient.

13. A method of examining a patient in order to provide an ultrasound treatment for that patient, comprising:
a) providing at least one upper audio frequency tone to the patient, to determine an optimum frequency for the patient; and
b) providing a plurality of audible frequencies modulated by the determined optimum frequency, so as to determine a particular audible frequency that is optimum for the patient with respect to tinnitus masking.

14. A method of suppressing tinnitus, comprising:
a) providing music by way of a first input;
b) providing at least one tone within a range of from 10 kHz to 20 kHz;
c) multiplying the music with the at least one tone to provide a tinnitus treatment signal; and
d) recording the tinnitus treatment signal onto a recording medium, for playback at a later time, so as to treat a patient by playing the tinnitus treatment signal from the recording medium.

15. The method according to claim 14, wherein the recording medium is a compact disk.

16. The method according to claim 14, wherein the recording medium is an analog player.

17. The method according to claim 14, wherein the recording medium is a digital player.

18. The method according to claim 14, wherein the at least one tone is noise within a range of from 10 kHz to 20 kHz.
